(19) United States
(12) Patent Application Publication
(10) Pub. No.: US 2024/0033237 A1
(43) Pub. Date: Feb. 1, 2024

(54) USE OF A-KETOGLUTARATE IN MANUFACTURE OF MEDICAMENT

(71) Applicant: TONGJI UNIVERSITY, Shanghai (CN)

(72) Inventors: Lei CUI, Shanghai (CN); Shuaijun LI, Shanghai (CN)

(73) Assignee: TONGJI UNIVERSITY, Shanghai (CN)

(21) Appl. No.: 17/787,950

(22) PCT Filed: Dec. 23, 2020
(86) PCT No.: PCT/CN2020/138554

§ 371 Date: Sep. 18, 2023

(30) Foreign Application Priority Data
Dec. 25, 2019 (CN) 201911353216.5

(51) Int. Cl. A61K 31/194; A61P 19/02

(52) U.S. Cl. CPC A61K 31/194; A61P 19/02

(57) ABSTRACT

The use of α-KG in the manufacture of a medicament, the medicament being used for: 1) treating osteoarthritis and related diseases; and/or, 2) inhibiting the catabolic phenotype of chondrocytes; and/or, 3) promoting the synthetic phenotype of chondrocytes; and/or, 4) promoting the regeneration of skin hair follicles. A pharmaceutical composition provided by the present disclosure can protect cartilage tissue and impede the progression of osteoarthritis, thus having great potential application for the clinical treatment and prevention of osteoarthritis. Further, the medicament provided by the present disclosure can significantly increase the number of skin hair follicles, which also has great potential application value for the treatment and improvement of hair loss.

### TECHNICAL FIELD

The present disclosure relates to the field of biotechnology, and in particular, to a use of α-ketoglutarate in the manufacture of a medicament.

### BACKGROUND

Osteoarthritis (OA) is a common chronic joint disease. The prevalence of osteoarthritis is high, ranking it as the sixth most disabling disease in the world. By 2020, osteoarthritis is expected to be the fourth most disabling disease. The onset of the disease is mostly seen in middle-aged and elderly people, more women than men. Osteoarthritis mostly occurs in a heavily loaded knee, ankle, spine, and frequently moving finger joints. The main pathological changes in osteoarthritis include degeneration of the articular cartilage. Clinical symptoms of osteoarthritis usually include joint pain, pressing pain, stiffness and functional limitation, with synovitis and secondary osteophyte formation. Osteoarthritis leads to destruction of articular cartilage, as well as subchondral bone sclerosis. These pathological changes are closely related to the painful symptoms of the patients' joints and cause joint mobility impairment and joint deformity, which severely affects the patients' quality of life, causes or exacerbates other comorbidities, and reduces life expectancy. In advanced stages of this disease, the articular cartilage will be extensively stripped and the subchondral bone will be directly stressed, resulting in loss of joint function and eventual disability. The cause of osteoarthritis is not fully understood; its development is a long-term, chronic, and progressive pathological process, with age, obesity, mechanical injury, and genetics being the main risk factors. Each year, more than 200 million people seek medical attention for symptoms associated with osteoarthritis. With the rapidly increasing elderly population, the socioeconomic impact of osteoarthritis will become more significant.

Most clinical treatments such as pharmacological pain relief, physical therapy and joint cavity injection can only relieve the symptoms of osteoarthritis but do not prevent the destruction of articular cartilage and the formation and development of secondary osteophyte. Articular cartilage is composed of chondrocytes and cartilage matrix. Chondrocytes are the only cells present in articular cartilage tissue and play an important role in maintaining, remodeling and repairing cartilage, a non-vascular tissue. Chondrocytes integrate and respond to signals from different types and intensities of biomechanical stimuli (shear force, stress and pressure), and remodel the extracellular matrix to adapt to mechanical stimuli through the secretion of synthetic and catabolic factors. In this process, chondrocytes achieve a dynamic balance in the cartilage matrix by balancing synthetic (production of type II collagen and proteoglycan) and catabolic processes (production of various enzymes that degrade matrix components). In summary, the imbalance between synthetic and catabolic processes in cartilage tissue leads to the development of osteoarthritis.

Alpha-ketoglutarate (α-KG) is an intermediate product of action in the tricarboxylic acid cycle and a downstream metabolite for the synthesis of various amino acids and proteins, such as glutamine (Gln). It has been shown that Gln-derived α-ketoglutarate regulates macrophage differentiation to the M2 phenotype and inhibits macrophage differentiation to the M1 phenotype via the histone demethylase JMJD3. Also, α-KG inhibits the formation of p-IKKα/β and suppresses the expression of induced inflammatory factors.

Currently, there are no reports of α-KG in the treatment of arthritis; therefore, providing a medicament containing α-KG has great potential application.

### SUMMARY

The present disclosure provides a use of alpha-KG in the manufacture of a medicament, the medicament being used for: 1) treating osteoarthritis and related diseases; and/or, 2) inhibiting the catabolic phenotype of chondrocytes; and/or, 3) promoting the synthetic phenotype of chondrocytes; and/or, 4) promoting the regeneration of skin follicles.

The present disclosure further provides a pharmaceutical composition.

A first aspect of the present disclosure provides a use of alpha-KG in the manufacture of a medicament, the medicament being used for: 1) treating osteoarthritis and related diseases; and/or, 2) inhibiting the catabolic phenotype of chondrocytes; and/or, 3) promoting the synthetic phenotype of chondrocytes; and/or, 4) promoting the regeneration of skin follicles.

Optionally, the α-KG is used to inhibit the activation and/or transduction of the NF-κB signaling pathway in osteocytes.

Optionally, the α-KG regulates the catabolic phenotype and/or synthetic phenotype of chondrocytes in an interleukin-1β (IL-1β) stimulated environment.

Optionally, the IL-1β is used to stimulate chondrocytes.

Optionally, the concentration of IL-1β is 1-15 ng/ml.

Optionally, the form of α-ketoglutarate in the medicament is dimethyl-α-ketoglutarate.

Optionally, the medicament is administered via intra-articular injection.

A second aspect of the present disclosure provides a pharmaceutical composition, which includes α-KG.

Optionally, the pharmaceutical composition further includes a pharmaceutically acceptable carrier.

As described above, the present disclosure provides a use of α-KG in the manufacture of a medicament, and a pharmaceutical composition. According to the use of the present disclosure, the medicament is used for: 1) treating osteoarthritis and related diseases; and/or, 2) inhibiting the catabolic phenotype of chondrocytes; and/or, 3) promoting the synthetic phenotype of chondrocytes; and/or, 4) promoting the regeneration of skin hair follicles. The present disclosure also discloses the regulation of α-KG on the phenotypes of chondrocytes under the stimulation of IL-1β. In addition, the present disclosure also established a model simulating osteoarthritis in vivo, which proved that the administration of α-KG via intra-articular injection could significantly inhibit the progression of osteoarthritis. Therefore, the pharmaceutical composition of the present disclosure can protect cartilage tissue and impede the progression of osteoarthritis, and has great potential application for the clinical treatment and prevention of osteoarthritis. Further, the medicament provided by the present disclosure can significantly increase the number of skin hair follicles, which also has great potential application value for the treatment and improvement of hair loss. Other features, advantages and effects can be referred to the content disclosed within the claims and specification of the present disclosure.

### BRIEF DESCRIPTION OF THE DRAWINGS

FIG. 1 shows changes in amino acid content in chondrocytes upon stimulation by IL-1β.

FIG. 2 shows the expressions of catabolic phenotype and synthetic phenotype of chondrocytes stimulated by IL-1β in the presence or absence of Gln.

FIG. 2A shows the expressions of chondrocyte catabolic genes MMP3, MMP13, ADAMTS5 and NOS2 during IL-1β stimulation to chondrocytes, as detected by q-PCR.

FIG. 2B shows the expressions of chondrocyte catabolic genes MMP3, MMP13 and NOS2 during IL-1β stimulation to chondrocytes, as detected by western blot.

FIGS. 2C-F shows the expressions of chondrocyte catabolic genes MMP3, MMP13, ADAMTS5 and NOS2 when the chondrocytes were stimulated by IL-1β for 6 h, 12 h, 24 h and 36 h, respectively, as detected by q-PCR.

FIG. 2G shows the expressions of chondrocyte catabolic genes MMP3, MMP13 and NOS2 when the chondrocytes were stimulated by IL-1β for 12 h and 24 h, respectively, as detected by western blot.

FIGS. 2H-J shows the expressions of chondrocyte synthetic genes SOX9, COL2A1 and ACAN when the chondrocytes were stimulated by IL-1β for 6 h, 12 h, 24 h and 36 h, respectively, as detected by q-PCR.

FIG. 2K shows the expressions of chondrocyte synthetic genes SOX9 and COL2A1 when the chondrocytes were stimulated by IL-1β for 12 h and 24 h, respectively, as detected by western blot.

FIG. 3 shows the metabolism of Gln in chondrocytes when the chondrocytes were stimulated by IL-1β for 6 h, 12 h, 24 h and 36 h, respectively.

FIG. 3A shows the expressions of Gln transporter genes SLC1A5, SLC38A2 and SLC7A5, glutamine synthase gene GS, and glutaminase gene GLS, as detected by q-PCR.

FIG. 3B shows the expressions of Gln transporter genes SLC1A5, SLC38A2 and SLC7A5, glutamine synthase gene GS, and glutaminase gene GLS, as detected by western blot.

FIG. 4 shows the content of α-KG, a downstream metabolite of Gln, in chondrocytes stimulated by IL-1β.

FIG. 4A shows the change of α-KG content in chondrocytes stimulated by IL-1β for 36 h.

FIG. 4B shows the expressions of genes involved in the generation of α-KG in chondrocytes stimulated by IL-1β for 36 h, as detected by q-PCR.

FIG. 4C shows the expressions of genes involved in the generation of α-KG in chondrocytes stimulated by IL-1β for 0 h, 12 h, 24 h and 36 h, respectively, as detected by q-PCR.

FIG. 5 shows the expressions of catabolic phenotypes of chondrocytes in Ctrl group, IL-1β group, and IL-1β+αKG group.

FIG. 5A shows the expressions of cartilage catabolic phenotypic genes as detected by whole-genome sequencing.

FIG. 5B shows the expressions of cartilage catabolic phenotypic genes MMP3, MMP13, ADAMTS5 and NOS2 in chondrocytes stimulated by IL-1β for 24 h, as detected by q-PCR.

FIG. 5C shows the expressions of cartilage catabolic phenotypic genes MMP3, MMP13, ADAMTS5 and NOS2 when the chondrocytes were stimulated by IL-1β for 24 h, as detected by western blot.

FIGS. 5D-G shows the expressions of cartilage catabolic phenotypic genes MMP3, MMP13, ADAMTS5 and NOS2 when the chondrocytes were stimulated by IL-1β for 6 h, 12 h, 24 h and 36 h, respectively, as detected by q-PCR.

FIG. 5H shows the expressions of cartilage catabolic phenotypic genes MMP3, MMP13, ADAMTS5 and NOS2 when the chondrocytes were stimulated by IL-1β for 6 h, 12 h, 24 h and 36 h, respectively, as detected by western blot assay.

FIG. 6 shows the expressions of synthetic phenotypes of chondrocytes in Ctrl group, IL-1β group, and IL-1β+αKG group.

FIG. 6A shows the expressions of cartilage synthetic phenotypic genes as detected by whole-genome sequencing.

FIGS. 6B-D shows the expressions of chondrocyte synthetic genes SOX9, COL2A1 and ACAN when the chondrocytes were stimulated by IL-1β for 6 h, 12 h, 24 h and 36 h, respectively, as detected by q-PCR.

FIG. 6E shows the expressions of chondrocyte synthetic genes SOX9 and COL2A1 when the chondrocytes were stimulated by IL-1β for 6 h, 12 h, 24 h and 36 h, respectively, as detected by western blot.

FIG. 7 shows the activation of NF-κB signaling pathway in chondrocytes of Ctrl group, IL-1β group, and IL-1β+αKG group.

FIG. 7A shows the expressions of NF-κB signaling pathway-related genes as detected by whole-genome sequencing.

FIG. 7B shows the expression of P65 in the nucleus of chondrocytes when the chondrocytes were stimulated by IL-1β for 0 min, 15 min, 30 min, and 60 min in the presence or absence of Gln, as detected by western blot.

FIG. 7C shows the expression of P65 in the nucleus of chondrocytes when the chondrocytes were stimulated by IL-1β for 0 min, 15 min, 30 min, and 60 min with and without supplementation of α-KG, as detected by western blot.

FIG. 8 shows the progression of osteoarthritis in the PBS group, DMM group, and DMM+αKG group.

FIG. 8A shows histological sections of Safranin O/fast green stained mouse knee joint.

FIG. 8B shows the OARSI score to quantitatively evaluate the damage degree of articular cartilage.

FIG. 9 shows the stained histological sections of the skin hair follicles on the back of posterior limbs of the mice.

### DETAILED DESCRIPTION

The embodiments of the present disclosure will be described below. Those skilled in the art can easily understand other advantages and effects of the present disclosure according to contents disclosed by the specification. The present disclosure may also be implemented or applied through other different specific implementation modes. Various modifications or changes may be made to all details in the specification based on different points of view and applications without departing from the spirit of the present disclosure.

Before further describing the specific embodiments of the present disclosure, it should be understood that the scope of protection of the present disclosure is not limited to the following specific embodiments; it should also be understood that the terms used in the embodiments of the present disclosure are just for describing the specific embodiments instead of limiting the scope of the present disclosure. In the present specification and claims, the singular forms “a”, “an” and “the” include the plural forms, unless specifically stated otherwise.

When the numerical values are given by the embodiments, it is to be understood that the two endpoints of each numerical range and any one between the two may be selected unless otherwise stated. Unless otherwise defined, all technical and scientific terms used in the present disclosure have the same meaning as commonly understood by one skill in the art. In addition to the specific method, equipment and material used in the embodiments, any method, equipment and material in the existing technology similar or equivalent to the method, equipment and material mentioned in the embodiments of the present disclosure may be used to realize the invention according to the grasp of the existing technology and the record of the invention by those skilled in the art.

A first aspect of the present disclosure provides a use of α-KG in the manufacture of a medicament, the medicament being used for: 1) treating osteoarthritis and related diseases; and/or, 2) inhibiting the catabolic phenotype of chondrocytes; and/or, 3) promoting the synthetic phenotype of chondrocytes; and/or, 4) promoting the regeneration of skin follicles.

In a specific embodiment of the present disclosure, the α-KG is used, for example, to inhibit the activation and/or transduction of the NF-κB signaling pathway in osteocytes. Further, the osteocytes may be, for example, chondrocytes in cartilage tissues. The concentration of the α-KG may be, for example, 3 to 20 mM. Further, to improve the effective inhibition to osteoarthritis, the concentration of the α-KG may be 3 to 10 mM, for example, 3 mM, 5 mM, 7 mM, or 8 mM. Further, the form of α-KG in the medicament is dimethyl-α-ketoglutarate, which enters the cell for regulatory effect.

In a specific embodiment of the present disclosure, the α-KG regulates the catabolic phenotype and/or synthetic phenotype of chondrocytes in an IL-1β-stimulated environment. Further, the IL-1β is used to stimulate chondrocytes, and the concentration of IL-1β is 1-15 ng/ml, such as 1 ng/ml, 5 ng/ml, or 10 ng/ml.

A second aspect of the present disclosure provides a pharmaceutical composition, which includes α-KG.

The pharmaceutical composition may further include a pharmaceutically acceptable carrier. The carriers may include various excipients and diluents that are not themselves essential active ingredients and are not unduly toxic upon application. The dosage to be considered in the administration of the pharmaceutical composition should depend on the frequency and pattern of administration, the age, gender, weight, and general condition of the subject being treated, the condition and severity of the disease, the route of administration, any accompanying diseases to be treated, and other factors apparent to those skilled in the art. Also, depending on the condition of the subject being treated and other pathological conditions, the pharmaceutical composition of the present disclosure may be administered or applied in combination with one or more other therapeutically active compounds or substances. In a specific embodiment of the present disclosure, the medicament or pharmaceutical composition may be administered via intra-articular injection.

The present disclosure also conducted experiments in which IL-1β stimulation inhibits chondrocytes, to study the metabolism of Gln within chondrocytes. In an embodiment of the present disclosure, the stimulation of chondrocytes by IL-1β inhibits the metabolism of Gln within chondrocytes and reduces the amount of Gln in chondrocytes, thus establishing that osteoarthritis is associated with the level of Gln content in chondrocytes.

The present disclosure also conducted experiments in which II-1β stimulation inhibits chondrocytes, to study the metabolism of the downstream metabolite α-KG of Gln in chondrocytes. In a specific embodiment of the present disclosure, IL-1β stimulates chondrocytes, and inhibits the expression of genes that produce α-KG in chondrocytes. The amount of α-KG in chondrocytes becomes less, thus providing insight that supplementation/complementation of α-KG can inhibit the progression of osteoarthritis.

The present disclosure also performs experiments on the effect of α-KG on the catabolic and synthetic phenotypes of chondrocytes, and on NF-κB signaling. In a specific embodiment of the present disclosure, by means of whole-genome sequencing, it was detected that α-KG significantly promoted the synthetic phenotype of chondrocytes and inhibited the catabolic phenotype of cartilage after α-KG was complemented. This result was verified by q-PCR and western blot. Furthermore, whole-genome sequencing also detected that upon IL-1β stimulation conditions, the expression of many genes in the NF-κB signaling pathway, such as P65 in the nucleus of the NF-κB signaling pathway, was suppressed after α-KG was complemented. Therefore, it was thus established that α-KG could inhibit the production of inflammation and could regulate the phenotype of chondrocytes by suppressing the NF-κB signaling pathway.

The present disclosure also conducted experiments in which an osteoarthritis model was established. In an embodiment of the present disclosure, α-KG was injected into the articular cavity, OARSI quantitative scoring was performed, and it was found that α-KG significantly inhibited the progression of osteoarthritis.

The present disclosure also conducted experiments in which the regeneration level of skin hair follicles was studied. In an embodiment of the present disclosure, α-KG was injected by intradermal injection, and the results were observed by HE staining. The results indicated that α-KG could significantly increase the number of skin hair follicles and can be used to alleviate and/or treat hair loss.

The present disclosure will be described in more detail in the following by means of specific embodiments. Unless otherwise stated, the experimental methods, detection methods, and preparation methods disclosed in the present disclosure all employ conventional techniques of molecular biology, biochemistry, chromatin structure and analysis, analytical chemistry, cell culture, recombinant DNA technology in the technical field and related fields. These techniques are well described in the prior literature.

The experimental methods without specific conditions noted in the following embodiments are based on conventional methods and conditions, or selected according to the products' instructions. The α-KG used in the present disclosure is dimethyl-α-KG. The dimethyl-α-KG enters cells to play a regulatory role. The dimethyl-α-KG is commercially available, and the rest of the reagents and raw materials used in the present disclosure are also commercially available.

### Embodiment 1: Culture of Mouse Chondrocytes

Mouse chondrocytes were obtained from 7-day-old C57BL/6 mice (Shanghai Slac laboratory animal Co., Ltd.), and the procedure was as follows: under aseptic surgical conditions, bilateral knee cartilages of C57 mice were carefully stripped and digested with 0.2% neutral collagenase (Serva, Germany) for 8 h; cell precipitates were obtained by centrifugation, and the cells were resuspended in modified Eagle medium (DMEM) (Gibco, USA) containing 10% FBS, inoculated in culture dishes, and cultured with high-sugar DMEM medium (containing 10% FBS, 100 U/mL penicillin and 100 mg/L streptomycin) at 37° C. in a 5% CO₂ constant temperature incubator. The medium was changed for the first time at 48 h. When the cell fusion rate was observed to reach 80%-90%, the cells were digested with trypsin (Gibco, USA) at a concentration of 0.25% and then subcultured. The cells were collected by centrifugation and resuspended, and the concentration was adjusted for inoculation in culture dishes. The cells were subcultured by the above method, and the obtained cells were labeled as P1, P2, P3 generations, etc.

### Embodiment 2: Effect of IL-1β on the Content Level of Glutamine in Chondrocytes Cultured in Glutamine-Containing Medium

### 1. Experimental Methods

### (1) Preparation of Standard Solutions

Nineteen amino acid standard substances were weighed accurately and prepared with water into a mixture so that the final concentrations of the nineteen amino acids in the mixture were 0.05 μg/mL, 0.2 μg/mL, 0.5 μg/mL, 1 μg/mL, 2 μg/mL, 5 μg/mL, 10 μg/mL, 20 μg/mL, 50 μg/mL, and 100 μg/mL (eleven gradients).

### (2) Sample Pretreatment

The samples were vortexed for 30 s, fast-frozen in liquid nitrogen; the quenched cell solution was dissolved at room temperature, vortexed for 30 s, and centrifuged at 800 g for 1 min; the supernatant was transferred into a centrifuge tube and placed on dry ice; the cells were reset with 500 μL methanol (−80° C), fast-frozen in liquid nitrogen, and subjected to repeated vortex and centrifugation operations; the supernatant was combined, blown dry with liquid nitrogen, added with 1000 μL of alanine-d4 isotope internal standard at a concentration of 1 μg/mL, vortexed for mixing well, and subjected to short centrifugation; 100 μL of the samples were added with 60 μL of concentrated hydrochloric acid: n-butanol (1:3), mixed well before short centrifugation (for shaking off the liquid droplets from the lid), placed at a constant temperature of 65° C. for 15 min for derivatization, subjected to short centrifugation, and volatilized at a temperature below 45° C.; 100 μL of 80% acetonitrile in water was added for redissolving, and the mixed solution was directly loaded into LC-MS for analysis.

### (3) LC-MS Detection Procedure

Chromatographic conditions: Column: ACQUITY UPLC® BEH C18 column (2.1×100 mm, 1.7 μm, Waters Corporation, USA); injection volume: 5 μL, column temperature: 40° C.; mobile phase A—acetonitrile containing 0.1% formic acid and 0.1% heptafluorobutyric acid, mobile phase B-0.1% formic acid in water; flow rate: 0.2 mL/min; gradient elution procedure: 0˜1.5 min, 5% A; 1.5˜2 min, 5˜20% A; 2˜7 min, 20˜30% A; 7˜8.5 min, 30˜98% A; 8.5˜10.5 min, 98% A; 10.5˜11min, 98˜5% A; 11˜12.5min, 5% A.

MS conditions: electrospray ionization (ESI) source, positive ionization mode. Ion source voltage: 3200 V, solvent temperature: 380° C., cone voltage: 20 V. Scanning was performed using multiple reaction monitoring (MRM). The ion pairs for quantitative analysis are shown in Table 1 below.

TABLE 1
Ion pairs for quantitative analysis of nineteen amino acids

| Substance | parent | Dwell | Cone | Collision |
|---|---|---|---|---|
| to be tested | ion/daughter ion | (s) | Volt | Energy |
| Gly | 132.10/76.00 | 0.02 | 15.0 | 10.0 |
| Ala | 146.10/90.00 | 0.02 | 15.0 | 10.0 |
| Val | 174.20/72.00 | 0.02 | 24.0 | 15.0 |
| Pro | 172.10/70.00 | 0.02 | 23.0 | 15.0 |
| Thr | 176.10/74.00 | 0.02 | 23.0 | 15.0 |
| Ile | 188.20/69.00 | 0.02 | 19.0 | 21.0 |
| Leu | 188.20/86.00 | 0.02 | 23.0 | 16.0 |
| Orn | 189.20/70.00 | 0.02 | 13.0 | 15.0 |
| Met | 206.10/104.00 | 0.02 | 20.0 | 11.0 |
| His | 212.10/110.00 | 0.02 | 20.0 | 9.0 |
| Phe | 222.20/120.00 | 0.02 | 26.0 | 13.0 |
| Arg | 231.20/70.00 | 0.02 | 19.0 | 13.0 |
| Tyr | 238.10/136.00 | 0.02 | 20.0 | 10.0 |
| Asp | 246.20/144.00 | 0.02 | 20.0 | 10.0 |
| Trp | 261.20/159.00 | 0.02 | 20.0 | 10.0 |
| GABA | 160.10/87.00 | 0.02 | 18.0 | 16.0 |
| Ser | 162.10/60.00 | 0.02 | 20.0 | 10.0 |
| Lys | 203.10/84.00 | 0.02 | 20.0 | 13.0 |
| Glu | 260.20/84.00 | 0.02 | 25.0 | 21.0 |
| Ala-d4 | 150.10/94.00 | 0.02 | 20.0 | 10.0 |

(4) Standard Curve

The linearity of the standard solutions was investigated by performing LC-MS assays on the concentration series of the standards, respectively, with the concentration of the standards as the horizontal coordinate and the peak area ratio as the vertical coordinate. The linear regression equation obtained for each amino acid is shown in Table 2. Correlation coefficient >0.99.

TABLE 2
Linear regression equations and quantification
limits for the nineteen amino acids

| Abbrevia- | | | |
|---|---|---|---|
| tion of | | Correlation | linearity |
| amino | | coefficient | range |
| acid | regression equation | (r) | (μg/mL) |
| Gly | Y = 0.00394743X + 1.40423 | 0.9983 | 0.50-20.00 |
| Ala | Y = 0.00243902X + 4.71354 | 0.9985 | 0.50-100.00 |
| Val | Y = 0.00750339X + 0.378487 | 0.9985 | 0.10-20.00 |
| Pro | Y = 0.0275459X + 13.7056 | 0.9990 | 0.50-20.00 |
| Thr | Y = 0.00189441X + 0.24206 | 0.9961 | 0.10-20.00 |
| Ile | Y = 0.000438188X − 0.0677954 | 0.9971 | 0.50-10.00 |
| Leu | Y = 0.0145015X + 0.138213 | 0.9998 | 0.05-50.00 |
| Orn | Y = 0.00199156X + 0.0250666 | 0.9949 | 0.05-5.00 |
| Met | Y = 0.00542667X − 7.60211 | 0.9969 | 2.00-100.00 |
| His | Y = 0.00151341X + 0.00620336 | 0.9973 | 0.10-50.00 |
| Phe | Y = 0.0109735X + 0.37543 | 0.9997 | 0.05-50.00 |
| Arg | Y = 0.000532467X + 0.21825 | 0.9966 | 0.50-50.00 |
| Tyr | Y = 0.00212273X + 0.51809 | 0.9977 | 0.50-20.00 |
| Asp | Y = 0.00562819X + 2.97028 | 0.9987 | 0.50-20.00 |
| Trp | Y = 0.000614033X − 0.657851 | 0.9915 | 2.00-50.00 |
| GABA | Y = 0.0250696X + 10.1602 | 0.9965 | 0.50-20.00 |
| Ser | Y = 0.00177477X + 0.434372 | 0.9999 | 0.50-20.00 |
| Lys | Y = 0.00126823X + 0.186635 | 0.9960 | 0.50-20.00 |
| Glu | Y = 0.0249964X + 11.3942 | 0.9996 | 0.50-20.00 |

### 2. Experimental Results

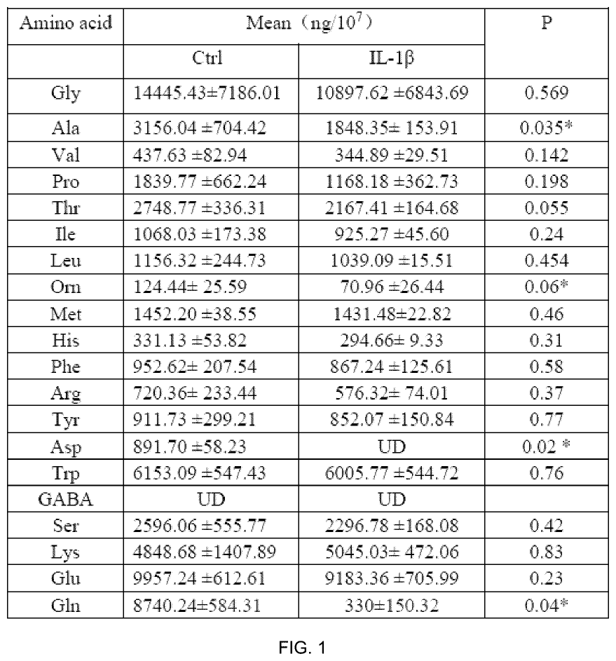

FIG. 1 shows changes of amino acid content in chondrocytes upon stimulation by IL-1β. After being treated with IL-1β for 36 h, the chondrocytes were scraped off, and the changes of amino acid content in the chondrocytes were detected by LC-MS. As shown in FIG. 1, it was found that upon the treatment of IL-1β, the expression of Gln decreased from 8740.24±584.31 ng/10⁷ to 330±150.32 ng/10⁷.

### Embodiment 3 Effect of IL-1β on Catabolic and Synthetic Phenotypes of Chondrocytes Cultured in Glutamine-Free Medium

### 1. Experimental Methods

### (1) Treatment of Mouse Chondrocytes

The P1 generation mouse chondrocytes cultured in Embodiment 1 were used in this Embodiment. After the P1 generation chondrocytes were grown adherent, they were cultured in serum-free DMEM culture medium containing 10 ng/ml IL-1β. For chondrocytes to be treated without glutamine, the chondrocytes were first changed to glutamamine-free medium 12 h in advance, and then treated with IL-1β.

(2) Real-Time Fluorescent Quantitative PCR (Real Time q-PCR) Analysis
1) The P1 generation mouse chondrocytes cultured in Embodiment 1 were used, the culture medium was sucked out from the culture dish, and the cells were washed once with PBS.
2) Extraction of total RNA
1. chloroform (⅕ volume of RNAiso Plus) was added to the homogenate lysate, the cover of the centrifuge tube was closed tightly, and the centrifuge tube was shaken violently by hand for 15 seconds. After the solution is fully emulsified (without phase separation phenomenon), the centrifuge tube was allowed to stand at room temperature for 5 minutes.
2. the centrifuge tube was centrifuged at 12000 g under 4° C. for 5 minutes.
3. the centrifuge tube was carefully taken out from the centrifugal machine, at this time the homogenate was divided into three layers: a colorless supernatant, a white protein layer in the middle, and a lower organic phase with color. The supernatant was transfered to a new centrifuge tube (do not suck out the white middle layer)
4. an equal volume of isopropanol was added to the supernatant, the new centrifuge tube was inverted up and down for mixing well, and then allowed to stand for 10 minutes at 15˜30° C.
5. the centrifuge tube was centrifuged at 12000 g under 4° C. for 10 minutes. Precipitate usually occurs at the bottom of a test tube after centrifugation.

### 3) Washing of RNA Precipitate

The supernatant was carefully discarded, 1 ml of 75% ethanol was slowly added along the wall of the centrifuge tube (do not touch the precipitate), the wall of the centrifuge tube was washed by gently turning the centrifuge tube upside down, the centrifuge tube was centrifuged at 12000 g under 4° C. for 5 minutes, and then the ethanol was carefully discarded (to better control the salt ion content in RNA, ethanol should be removed as much as possible).

### 4) Dissolution of RNA

The precipitate was dried at room temperature for 2˜5 minutes, an appropriate amount of RNase-free water was added to dissolve the precipitate, and if necessary, a pipette may be used to gently pipet the precipitate. After the RNA precipitate was completely dissolved, the concentration was immediately measured, and reverse transcription was carried out. The remaining RNA was stored at −80° C.

### 5) Measurement of RNA Concentration

The RNA samples obtained in (4) were measured for determining concentrations by Nanodrop 2000 instrument.

### 6) Reverse Transcription Reaction

The RNA samples obtained in (5) were subjected to a reverse transcription reaction, in which the reaction system and reaction conditions are shown in Table 3.

TABLE 3
Reaction system and reaction conditions of the reverse transcription
Reaction system (Reverse Transcription (RT)
reaction solution was prepared on ice)

| Reagent | Amount | |
|---|---|---|
| 5 × PrimeScript RT Master Mix | 2 | μl |
| Total RNA | 500 | ng |
| RNase Free dH₂O | up to 10 μl | |
| Total | 10 | μl |

Reaction conditions

| | | | |
|---|---|---|---|
| 37° C. | 15 | min | Reverse transcription reaction |
| 85° C. | 5 | sec | Inactivation of reverse transcriptase |

| | |
|---|---|
| 4° C. | ∞ |

### 7) RT-PCR Reaction

On ice, SYBR Premix Ex Taq, upstream and downstream primers of catabolic and synthetic genes of chondrocytes, ROX Reference Dye, cDNA template and dH2O were added into a test tube. After being mixed evenly, the mixture was evenly divided into PCR tubes. The PCR tubes were placed in a Q-PCR instrument to detect the product. The reaction system and reaction conditions for real-time PCR are shown in Table 4. Real-time PCR primer sequences are shown in Table 5.

TABLE 4
Reaction system and reaction conditions for real-time PCR
Reaction system (prepared on ice)

| Reagent | Amount | |
|---|---|---|
| SYBR Premix Ex Taq | 10 | μl |
| Primer F (10 uM) | 0.4 | μl |
| Primer R (10 uM) | 0.4 | μl |
| ROX Reference Dye | 0.4 | μl |
| cDNA template | 1.6 | μl |
| dH2O | 6.8 | μl |
| Total | 20 | μl |

Reaction conditions

| | | | | |
|---|---|---|---|---|
| Stage 1 Initial denaturation | 95° C. | 30 | s | Reps 1 |
| Stage 2 PCR reaction | 95° C. | 5 | s | Reps 40 |
| | 60° C. | 31 | s | |
| Stage 3 | 95° C. | 15 | s | Reps 1 |
| | 60° C. | 1 | min | |
| | 95° C. | 15 | s | |

TABLE 5
Real-time PCR primer sequences

| Gene | Primer (F = forward; R = reverse) | Amplicon size (bp) |
|---|---|---|
| Sox 9 | F: 5′-AGTACCCGCATCTGCACAAC-3′ (SEQ | 187 |
| | ID NO: 1) | |
| | R: 5′-ACGAAGGGTCTCTTCTCGCT-3′ (SEQ | |
| | ID NO: 2) | |
| COL2A1 | F: 5′-AGCAAGGTGACCAGGGTATT-3′ (SEQ | 98 |
| | ID NO: 3) | |
| | R: 5′-ACCAGGAGAGCCACGTTC-3′ (SEQ ID | |
| | NO: 4) | |
| ACAN | F: 5′-CGTTGCAGACCAGGAGCAAT-3′ (SEQ | 275 |
| | ID NO: 5) | |
| | R: 5′-GGTTTGGACGCCACTTCTCA-3′ (SEQ | |
| | ID NO: 6) | |
| MMP3 | F: 5′-TCCTGATGTTGGTGGCTTCAG-3′ (SEQ | 102 |
| | ID NO: 7) | |
| | R: 5′-TGTCTTGGCAAATCCGGTGTA-3′ (SEQ | |
| | ID NO: 8) | |
| MMP13 | F: 5′-ACTACCATCCTGCGACTCTTG-3′ (SEQ | 111 |
| | ID NO: 9) | |
| | R: 5′-GTTTGCCAGTCACCTCTAAGC-3′ (SEQ | |
| | ID NO: 10) | |
| NOS2 | F: 5′-ACCTTGTTCAGCTACGCCTT-3′ (SEQ | 112 |
| | ID NO: 11) | |
| | R: 5′-CATTCCCAAATGTGCTTGTC-3 (SEQ ID | |
| | NO: 12) | |
| GLS | F: 5′-AACGTCAGATGGTGTCATGCT-3′ | 206 |
| | (SEQ ID NO: 13) | |
| | R: 5′-TGAATTTGGCCAGCTGAGGA-3′ (SEQ | |
| | ID NO: 14) | |
| SLC1A5 | F: 5′-TCGTCTTTGGTGTGGCTCTG-3′ (SEQ | 176 |
| | ID NO: 15) | |
| | R: 5′-CTGGCGGACGTCTTTCATCT-3′ (SEQ | |
| | ID NO: 16) | |
| SLC7A5 | F: 5′-CTTCGCCACCTACTTGCTCA-3′ (SEQ ID | 225 |
| | NO: 17) | |
| | R: 5′-CCTTGTCCCATGTCCTTCCC-3′ (SEQ ID | |
| | NO: 18) | |
| SLC38A2 | 5′-ATTGTGGGCAGTGGAATCCTT-3′ (SEQ ID | 134 |
| | NO: 19) | |
| | 5′-TCGTTGGCAGTCTTGAGGAG-3′ (SEQ ID | |
| | NO: 20 | |
| GS | 5′-CTGGGTTGATGGTACCGGAG-3′ (SEQ ID | 177 |
| | NO: 21) | |
| | 5′-CGGAAGGGGTCTCGAAACAT-3′ (SEQ ID | |
| | NO: 22) | |
| GLUD1 | 5′-CAATGCGCATGCCTGTGTTA-3′ (SEQ ID | 205 |
| | NO: 23) | |
| | 5′-CATAGAGTGCAGGCCCACAT-3′ (SEQ ID | |
| | NO: 24) | |
| IDH1 | 5′-CAGAGCTCTCTTGGACCGAC-3′ (SEQ ID | 247 |
| | NO: 25) | |
| | 5′TGCAGCATCTTTGGTGACCT-3′ (SEQ ID | |
| | NO: 26) | |
| IDH2 | 5′-AAGTCTTCCGGTGGCTTTGT-3′ (SEQ ID | 182 |
| | NO: 27) | |
| | 5′-TGGTGTTCTCGGTAATGGCG-3′ (SEQ ID | |
| | NO: 28) | |
| GOT1 | 5′-AGAGCAGTGGAAGCAGATCG-3′ (SEQ ID | 122 |
| | NO: 29) | |
| | 5′-AATAGCGAATAGCCCACGCA-3′ (SEQ ID | |
| | NO: 30) | |
| GOT2 | 5′-GGACCTCCAGATCCCATCCT-3′ (SEQ ID | 107 |
| | NO: 31) | |
| | 5′-GGTTTTCCGTTATCATCCCGGTA-3′ (SEQ | |
| | ID NO: 32) | |
| CCBL2 | 5′-TTCAAAAACGCCAAACGAATCG-3′ (SEQ | 196 |
| | ID NO: 33) | |
| | 5′-GATGACCAAAGCCTCTTGTGT-3′ (SEQ ID | |
| | NO: 34) | |
| GPT2 | 5′-AACCATTCACTGAGGTAATCCGA-3′ (SEQ | 121 |
| | ID NO: 35) | |
| | 5′-GGGCTGTTTAGTAGGTTTGGGTA-3′ (SEQ | |
| | ID NO: 36) | |
| IDH3a | 5′-TGGGTGTCCAAGGTCTCTC-3′ (SEQ ID | 177 |
| | NO: 37) | |
| | 5′-CTCCCACTGAATAGGTGCTTTG-3′ (SEQ | |
| | ID NO: 38) | |
| IDH3b | 5′-TGGAGAGGTCTCGGAACATCT-3′ (SEQ ID | 150 |
| | NO: 39) | |
| | 5′-AGCCTTGAACACTTCCTTGAC-3′ (SEQ ID | |
| | NO: 40) | |
| IDH3g | 5′-GGTGCTGCAAAGGCAATGC-3′ (SEQ ID | 124 |
| | NO: 41) | |
| | 5′-TATGCCGCCCACCATACTTAG-3′ (SEQ ID | |
| | NO: 42) | |

### (3) Western Blot Analysis

P1 generation mouse chondrocytes cultured in Embodiment 1 were subjected to western blot analysis through operations including the following:
1. Collecting of protein samples: The culture medium was sucked out, and the cells were washed twice with PBS at 4° C. and then dried. 100 μl of cell lysate was added to each well, the cells were lysed on ice for 10 min, and collected into a 1.5 ml centrifuge tube. The centrifuge tube was centrifuged at 10,000 rpm for 10 min, and the supernatant was collected. The protein concentration was determined by the BCA Protein Quantification Kit. The protein was mixed with 5× loading buffer, heated at 100° C. for 10 min, and stored at −80° C. for later use.
2. Electrophoresis: SDS-PAGE gel was configured according to protein size, and 20 μg sample was added per well. After running at a constant voltage of 80 volts until reaching separating gel, the voltage was adjusted to 120V, and electrophoresis could be stopped after protein bands were separated according to protein marker.
3. Protein transfer from gel to membrane: pre-cut PVDF membrane and filter paper were activated in methanol for 5 min, then rinsed in transfer buffer for later use. The gel block was taken from the glass plate and the running buffer was washed off with water. The transfer system was composed in the following order: cathode splint, sponge, filter paper, gel, PVDF membrane, filter paper, sponge, and anode splint. The transfer apparatus was placed in an ice box and added with transfer buffer. The gel is close to the cathode, and the PVDF membrane is close to the anode. The transferring was performed at 100V for 1.5 h.
4. Blocking: the PVDF membrane was removed and soaked in 5% skim milk. The 5% skim milk was prepared with 1×TBST, 500 μl tween 20 was dissolved in 500 ml ×TBS to form TBST. The blocking was performed at room temperature for 1 h.
5. Primary antibody incubation: After blocking, the blocking buffer was discarded, and the membrane was washed with 1×TBST, 5 min/time, 5 times. The primary antibody was diluted according to the concentration of primary antibody dilution, and then incubated at 4° C. overnight.
6. Secondary antibody incubation: the primary antibody was recovered; the membrane was washed with 1×TBST, 5 min/time, 5 times; the secondary antibody was incubated for 1 h at room temperature in a shaker.
7. Membrane scan for development: the membrane was exposed and scanned by a chemiluminescence system.

### (4) Extraction of Nuclear Protein

P1 generation mouse chondrocytes cultured in Embodiment 1 were used to extract nuclear proteins for observing changes of P65 in the NF-κB signaling pathway by a method including the following:
1. Reagents were placed on ice and mixed well. An appropriate amount of cytoplasmic protein extraction reagent I was taken, and added with PMSF and protease inhibitor within 1 min before use. An appropriate amount of nuclear protein lysate was taken, and added with PMSF and protease inhibitor within 1 min before use.
2. The cells were washed once with PBS.
3. The cells were treated with EDTA solution, and pipetted with a pipette. The cells were collected by centrifugation, and the supernatant was sucked out to obtain cell precipitates.
4. 200 μL of cytoplasmic protein extraction reagent I containing PMSF was added for every 20 μL of cell precipitates.
5. The mixture was shaken violently at the highest speed for 10 s to completely disperse the cell precipitates.
6. The mixture was bathed in ice for 10-15 minutes.
7. 11 μl of cytoplasmic protein extraction reagent II was added. The mixture was shaken violently at the highest speed for 5 seconds and placed on ice for 1 min.
8. The mixture was shaken violently at the highest speed for 5 seconds, and centrifuged at 16,000 g under 4° C. for 5 min.
9. The supernatant was immediately transfered into a pre-cooled 1.5 ml centrifuge tube, namely cytoplasmic protein.
10. The remaining supernatant was sucked out completely. 50 μL of nuclear protein lysate containing PMSF was added.
11. The mixture was shaken violently at the highest speed for 15-20 seconds and placed on ice. The mixture was violently shaken at high speed for 15-30 s every 10 min, 4 times in total.
12. The mixture was centrifuged at 16,000 g under 4° C. for 10 min.
13. The supernatant was immediately transfered into a pre-cooled 1.5 ml centrifuge tube, namely the extracted nuclear protein, which was frozen at −70° C. for later use.

### 2. Experimental Results

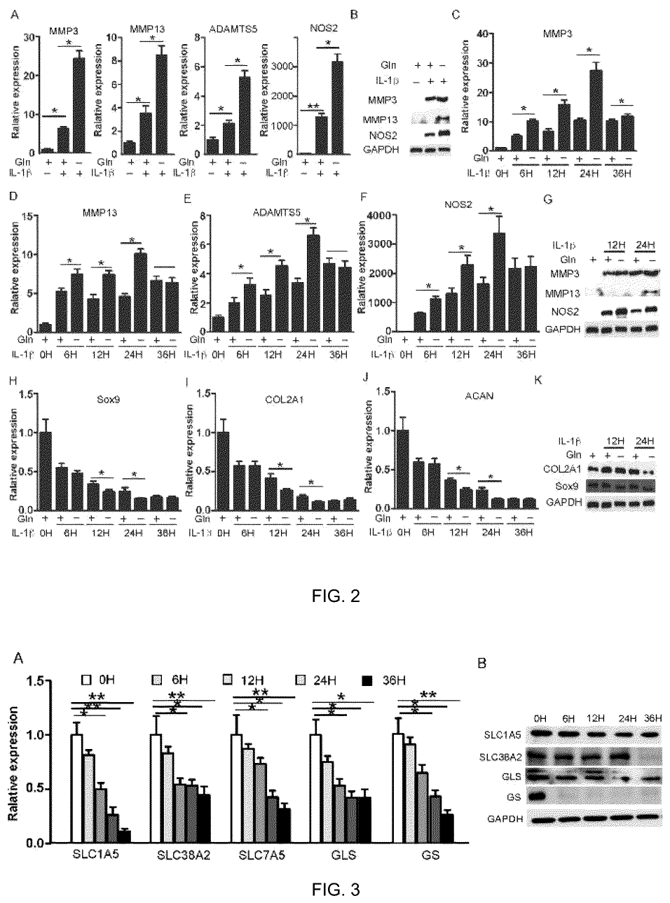

FIG. 2 shows the catabolic phenotype and synthetic phenotype of chondrocytes stimulated by IL-1β in Gln-deficient conditions. As shown in FIG. 2, q-PCR detection results indicated that chondrocytes stimulated by IL-1β in Gln-free conditions had significantly higher expression of catabolic phenotypic genes (including MMP3, MMP13, ADAMTS5, and NOS2) than those stimulated by IL-1β in Gln-containing conditions (FIG. 2A). After the chondrocytes were stimulated by IL-1β for 24 h, the protein expression of the catabolic phenotype was consistent with the gene expression, that is, the protein expression of the catabolic phenotype was higher in the absence of Gln (FIG. 2B). The expression of cartilage catabolic phenotypic genes such as MMP3, MMP13, ADAMTS5 and NOS2 were found to be higher in the absence of Gln than in the presence of Gln at various time periods, such as 6 h, 12 h, 24 h and 36 h after IL-1β stimulation (FIGS. 2C-F). At the protein level, western blot analysis also found that the stimulation to chondrocytes by IL-1β in the absence of Gln promoted the protein expression of cartilage catabolic phenotypic genes, such as MMP3, MMP13 and NOS2, at different time points (FIG. 2G). For synthetic genes of chondrocytes, such as SOX9, COL2A1 and ACAN, q-PCR detection results indicated that the expression of SOX9, COL2A1 and ACAN decreased after the chondrocytes were stimulated by IL-1β for 12 h and 24 h in the absence of Gln (FIGS. 2H-J), but there was no significant difference at 6 h and 36 h. This may be due to the inhibition of the glutamine transporter after IL-1β stimulation, and the difference between with and without glutamine was not significant at later stages. At the protein level, western blot analysis found that the stimulation to chondrocytes by IL-1β for 36 h in the absence of Gln decreased the expression of SOX9 and COL2A1 proteins, compared to that in the presence of Gln (FIG. 2K).

FIG. 3 shows metabolism of Gln in chondrocytes upon stimulation by IL-1β. Under the condition of IL-1β stimulation, the expression of Gln transporter gene in chondrocytes was detected by Q-PCR. As shown in FIG. 3, it was found that IL-1β stimulation resulted in the decreased expression of Gln transporter genes such as SLC1A5, SLC38A2 and SLC7A5. This phenomenon was time-dependent. The longer the stimulation, the lower the expression of these genes (FIG. 3A). Further, expressions of glutaminase (GLS) and glutamine synthetase (GS) were also detected. Q-PCR and western blot results showed that the expressions of GLS and GS decreased significantly, and the longer the stimulation, the lower their expressions (FIGS. 3A-B).

### Embodiment 4 Effect of α-ketoglutarate on the Catabolic and Synthetic Phenotypes of Chondrocytes Cultured in Glutamine-Containing Cultures

### 1. Experimental Methods

### (1) Treatment of Mouse Chondrocytes

Mouse chondrocytes of P1 generation obtained in Embodiment 1 were cultured in blank DMEM culture medium, DMEM culture medium containing 10 ng/ml IL-1β, and DMEM culture medium containing 10 ng/ml IL-1β and 7 mM α-KG, respectively, to obtain the corresponding Ctrl group, IL-1β group, and IL-1β+αKG group.

(2) Real-Time Fluorescent Quantitative PCR (Real Time q-PCR) Analysis

The Ctrl group, IL-1β group and IL-1β+αKG group were subjected to q-PCR assay under the same experimental conditions as in Embodiment 3, respectively.

### (3) Western Blot Analysis

The Ctrl group, IL-1β group and IL-1β+αKG group were subjected to western blot assay under the same experimental conditions as in Embodiment 3, respectively.

### 2. Experimental Results

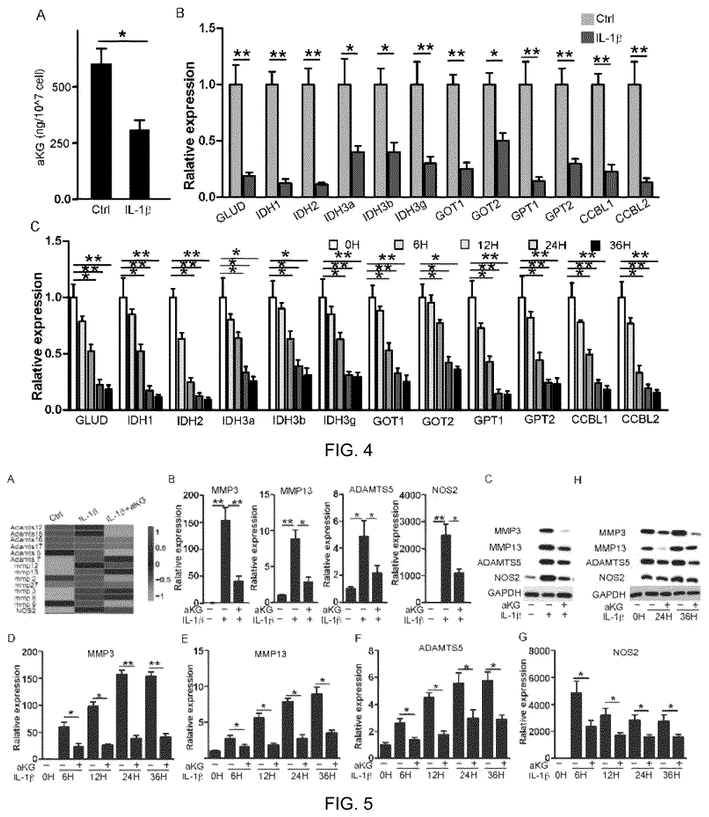

FIG. 4 shows the content of α-KG, a downstream metabolite of Gln, in chondrocytes stimulated by IL-1β. As shown in FIG. 4, the content of α-KG in chondrocytes in the IL-1β group decreased significantly compared with that in the Ctrl group, that is, the content of α-KG in chondrocytes decreased after IL-1β stimulation (FIG. 4A). Furthermore, expressions of a-KG related genes such as DLUD, IDH1, IDH2, IDH3a, IDH3b, IDH3g, GOT1, GOT2, GPT1, GPT2, CCBL1 and CCBL2 were detected by Q-PCR. It was found that the expressions of all these genes were inhibited after IL-1β stimulation for 36 h (FIG. 4B). This phenomenon was time-dependent, i.e., the longer the stimulation, the lower the expression of these genes (FIG. 4C).

FIG. 5 shows the expressions of catabolic phenotypes of chondrocytes in Ctrl group, IL-1β group, and IL-1β-30 αKG group. The whole-genome expression was detected by whole-genome sequencing (the whole-genome sequencing service was provided by Shanghai Biotechnology Corporation). As shown in FIG. 5, expressions of catabolic phenotypes (such as metalloproteases MMP2, MMP3, MMP8, MMP9, MMP12, MMP13, MMP27; and aggrecanases ADAMTS5, ADAMTS7, ADAMTS12, ADAMTS15, ADAMTS16, ADAMTS17; and NOS2, which induces apoptosis in cartilage) of chondrocytes in the IL-1β group were increased compared to those in the Ctrl group (FIG. 5A). Meanwhile, the expressions of catabolic phenotypes (such as metalloproteases MMP2, MMP3, MMP8, MMP9, MMP12, MMP13, MMP27; and aggrecanases ADAMTS5, ADAMTS7, ADAMTS12, ADAMTS15, ADAMTS16, ADAMTS17; and NOS2, which induces apoptosis in cartilage) of chondrocytes in the IL-1β+αKG group were suppressed. To further validate the results of the whole-genome sequencing, the expressions of cartilage catabolic phenotypic genes such as MMP3, MMP13, ADAMTS5, and NOS2 were analyzed by q-PCR after the chondrocytes were treated with IL-1β, IL-1β+α-KG respectively for 24 h. The results showed that α-KG could reverse the effect of IL-1β and inhibit the expression of cartilage catabolic phenotypic genes MMP3, MMP13, ADAMTS5, and NOS2 (FIG. 5B). The expressions at the protein level were analyzed by western blot assay, and it was found that α-KG inhibited the protein expression of cartilage catabolic phenotypic genes such as MMP3, MMP13, ADAMTS5 and NOS2 (FIG. 5C). Moreover, q-PCR and western blot results revealed that replenishing α-KG at different time periods, such as 6 h, 12 h, 24 h, and 36 h, could inhibit the gene and protein expressions of the catabolic phenotypic genes MMP3, MMP13, ADAMTS5, and NOS2 in chondrocytes (FIGS. 5D-H).

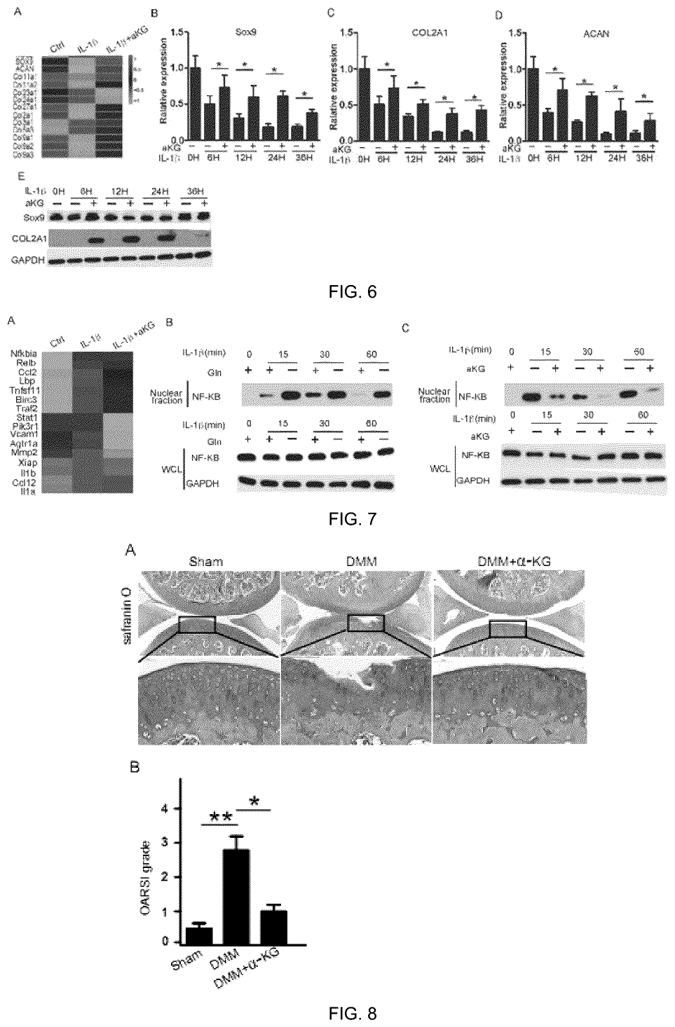

FIG. 6 shows the expressions of synthetic phenotypes of chondrocytes in Ctrl group, IL-1β group, and IL-1β+αKG group after 24 h of treatment. The whole-genome expression was detected by whole-genome sequencing. As shown in FIG. 6, expressions of synthetic phenotypic genes (such as SOX9, COL2A1, ACAN and some other collagen of cartilage, such as COL9A1, COL9A2, COL9A3, COL11A1, and COL11A2) of chondrocytes in the IL-1β group were decreased, and the expression of the cartilage dedifferentiation gene COL3A1 in the IL-1β group was increased, compared to those in the Ctrl group. Meanwhile, the expressions of synthetic phenotypic genes (such as SOX9, COL2A1, ACAN and some other collagen of cartilage, such as COL9A1, COL9A2, COL9A3, COL11A1, and COL11A2) of chondrocytes in the IL-1β+αKG group were increased. The results showed that after replenishing α-KG, α-KG could reverse the effect of IL-1β (FIGS. 6A). To further validate the results of the whole-genome sequencing, expressions of synthetic genes of chondrocytes in the IL-1β+αKG group, such as SOX9, COL2A1, and ACAN, were analyzed by q-PCR assay. It was found that replenishing α-KG could promote the expressions of chondrocyte synthetic genes SOX9, COL2A1, and ACAN while the chondrocytes were stimulated by IL-1β (FIGS. 6B-D). The expressions at the protein level of chondrocytes in the IL-1β+αKG group were analyzed by western blot assay, and it was found that replenishing α-KG could promote the protein expressions of chondrocyte synthetic genes such as SOX9 and COL2A, and similar results were found at 6 h, 12 h, 24 h, and 36 h (FIG. 6E).

FIG. 7 shows the activation of NF-κB signaling pathway in chondrocytes of Ctrl group, IL-1β group, and IL-1β+αKG group. The whole-genome expression was detected by whole-genome sequencing. As shown in FIG. 7, expressions of NF-κB signaling pathway-related genes of chondrocytes in the IL-1β group, such as Nfkbia, Relb, Lbp, Pik3r1, Tnfsf11, Birc3, Traf2, Stat1, MMP2, xiap, Ccl2, Ccl12, IL-1β, and IL-1α, were inhibited (FIG. 7A). To further verify whether the NF-κB signaling pathway could be activated in the absence of glutamine, chondrocytes were cultured without glutamine for 12 h, followed by stimulation with IL-1β for 0 min, 15 min, 30 min, and 60 min, and it was found that the expression of P65 in the NF-κB signaling pathway was significantly increased in the absence of glutamine (FIG. 7B). Meanwhile, the expression of P65 in the NF-κB signaling pathway in the nucleus of chondrocytes of the IL-1β+αKG group at 0 min, 15 min, 30 min, and 60 min was observed. The results showed that at 0 min, 15 min, 30 min and 60 min, the expression of P65 in the NF-κB signaling pathway in the nucleus was significantly inhibited in the chondrocytes of IL-1β+αKG group (FIG. 7C). This indicates that the NF-κB signaling pathway was inhibited by supplementation of α-ketoglutarate when the chondrocytes were stimulated by IL-1β.

### Embodiment 5 Mouse Model of Knee Osteoarthritis (DMM)

### 1. Experimental Methods

### (1) Construction of Mouse Knee DMM Model

Male C57BL/6 mice (n=30) were purchased from Shanghai Slac laboratory animal Co., Ltd. and reared to 8 weeks of age. The animal experimental operation method was reviewed and approved by the Ethics Committee of Tongji University School of Medicine.

Male C57BL/6 mice reared to 8 weeks of age were anaesthetized, and a longitudinal incision was made in the medial side of the knee joint. The articular cavity was opened along the medial side of the patellar ligament, the fat pad in the intercondylar area was bluntly separated, and the meniscus tibial ligament connecting the medial meniscus was found and transected. The incision was closed after hemostasis by compression, and the mice were continued to be fed. The mice were anesthetized and sacrificed at 4, 8 and 12 weeks after surgery (n=8 for each group), and the knee joints were isolated for histological examination, forming the treatment group.

Male C57BL/6 mice reared to 8 weeks of age were anaesthetized, and a longitudinal incision was made in the medial side of the knee joint. The articular cavity was opened along the medial side of the patellar ligament, the fat pad in the intercondylar area was bluntly separated, and the meniscus tibial ligament connecting the medial meniscus was found, exposed, but not cut. The incision was closed after hemostasis by compression, and the mice were continued to be fed. The mice were anesthetized and sacrificed at 4, 8 and 12 weeks after surgery (n=8 for each group), and the knee joints were isolated for histological examination, forming the sham group.

(2) Intra-Articular Injection of α-KG into the Knee Joint of Mice for Histological Staining

Three weeks after DMM surgery, intra-articular injections were given to the mice in the treatment group and sham group, once a week, 5 times in total. After the mice were anesthetized, the skin at the knee joint was wiped with alcohol for disinfection, and the articular cavity of the treatment group was injected with 10 μL of PBS-diluted α-KG (1 μl dimethyl-α-KG dissolved in 9 μl PBS) using an insulin needle. A sham operation was performed and PBS was injected as a control. After 5 injections, i.e., 8 weeks after DMM surgery, samples were collected, and histological staining was performed to observe the changes of articular cartilage.

### (3) Histological Detection and Evaluation

3 weeks after the DMM surgery, mice in sham group was injected with PBS, mice in a DMM group was injected with PBS, and mice in a DMM+α-KG group was injected with α-ketoglutarate. The corresponding mouse knee joints were taken, and treated by the following methods to form specimens:
1. Mouse knee joints were fixed in 4% paraformaldehyde for 24 h. The knee joints were decalcified in 10% EDTA in a shaker for 10 d.
2. The knee joints were paraffin-embedded. Coronal sections were made in the anterior middle of the tibial plateau, or sagittal sections were made in the medial part of the tibial plateau.
3. The sections were stained with HE or with Safranin-O/fast green.

Safranin-O/fast green staining includes the following procedures:
1. Deparaffinizing the paraffin sections to water.
2. Staining with Safranin-O for 20 min.
3. Rinsing once with 1% acetic acid.
4. Staining with fast green for 3 min.
5. Washing with water, dehydrating in gradient alcohol, clearing in xylene, and mounting.

The damage degree of articular cartilage was observed by Safranin-O/ fast green staining and quantitatively evaluated by OARSI score. The evaluation criteria are shown in Table 6 below.

TABLE 6
Recommended semi-quantitative scoring system for the damage degree of
osteoarthritis

| Damage degree | Appearance |
|---|---|
| 0 | Normal |
| 0.5 | Loss of Staining without structural changes |
| 1 | Small fibrillations without loss of cartilage |
| 2 | Vertical clefts down to the layer immediately below |
| | the superficial layer and some loss of surface lamina |
| 3 | Vertical clefts/erosion to the calcified cartilage |
| | extending to <25% of the articular surface |
| 4 | Vertical clefts/erosion to the calcified cartilage extending |
| | to 25-50% of the articular surface |
| 5 | Vertical clefts/erosion to the calcified cartilage extending |
| | to 50-75% of the articular surface |
| 6 | Vertical clefts/erosion to the calcified cartilage |
| | extending >75% of the articular surface |

### (4) Statistical Analysis

The scores of sham group, DMM group, and DMM+αKG group were tested by unpaired samples t-test. The statistical software used was SPSS11.0, and P<0.05 was considered statistically different.

### 2. Experimental Results

FIG. 8 shows the effect of α-KG on inhibiting the progression of osteoarthritis (the osteoarthritis was simulated by DMM surgery). As shown in FIG. 8, eight weeks after the DMM surgery, the DMM group showed severe joint destruction compared with the PBS group, and the progression of osteoarthritis was obvious. Meanwhile, the DMM+αKG group showed no significant cartilage damage and the progression of osteoarthritis was not obvious (FIG. 8A). Meanwhile, the OARSI score was used to quantitatively evaluate the damage degree of articular cartilage, and it was found that α-KG could significantly inhibit the progression of osteoarthritis (FIG. 8B).

### Embodiment 6 Regeneration Level of Skin Hair Follicles in Mice

### (1) Experimental Methods

The skin on the back of posterior limbs of 12-month-old aged mice was taken, and the treatment group was intradermally injected with PBS-diluted α-KG (1 μl of dimethyl-α-KG dissolved in 4 μl of PBS). A sham operation was performed and PBS was injected as a control. Histological staining was performed to observe the changes of skin hair follicles at 4 weeks after injection.

### (2) Experimental Results

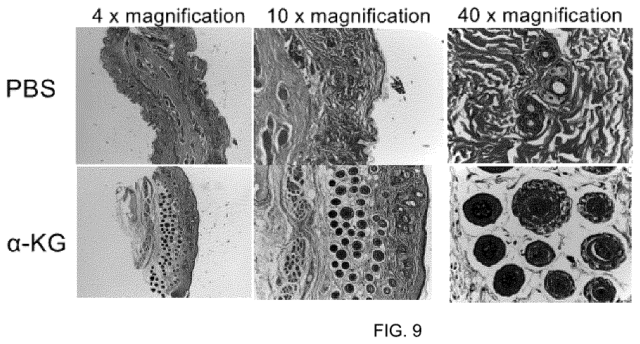

FIG. 9 shows the stained histological sections of the skin hair follicles on the back of posterior limbs of mice to observe the effect of α-KG on promoting the regeneration of skin hair follicles. As shown in FIG. 9, after 4 weeks, compared with the PBS group, the number of skin hair follicles of the mice injected with dimethyl-α-ketoglutarate was significantly increased.

In summary, the present disclosure effectively overcomes various shortcomings and has high industrial utilization value. The above-mentioned embodiments are just used for exemplarily describing the principle and effects of the present disclosure instead of limiting the present disclosure. Modifications or variations of the above-described embodiments may be made by those skilled in the art without departing from the spirit and scope of the present disclosure. Therefore, all equivalent modifications or changes made by those who have common knowledge in the art without departing from the spirit and technical concept disclosed by the present disclosure shall be still covered by the claims of the present disclosure.

1. The use of dimethyl-α-ketoglutarate in the manufacture of a medicament, wherein the medicament is used for:
1) treating osteoarthritis and related diseases; and/or,
2) inhibiting a catabolic phenotype of chondrocytes; and/or,
3) promoting a synthetic phenotype of chondrocytes.

2. The use according to claim 1, wherein the dimethyl-α-ketoglutarate is used to inhibit the activation and/or transduction of NF-κB signaling pathway in osteocytes.

3. The use according to claim 1, wherein the dimethyl-α-ketoglutarate regulates the catabolic phenotype and/or the synthetic phenotype of chondrocytes in an interleukin-1β-stimulated environment.

4. The use according to claim 3, wherein the interleukin-1β stimulates the chondrocytes.

5. The use according to claim 3, wherein a concentration of the interleukin-1β is 5-15 ng/ml.

6. (canceled)

7. The use according to claim 1, wherein the medicament is administered via intra-articular injection.

8. A pharmaceutical composition, wherein the pharmaceutical composition comprises dimethyl-α-ketoglutarate.

9. The pharmaceutical composition according to claim 8, further comprising a pharmaceutically acceptable carrier.

10. A method of treating osteoarthritis and related diseases, comprising administering a therapeutically effective amount of the pharmaceutical composition of claim 8 to a subject in need thereof.

11. A method of treating osteoarthritis and related diseases, comprising administering a therapeutically effective amount of the pharmaceutical composition of claim 8 to a subject in need thereof via intra-articular injection.


SEQUENCE LISTING (42 sequences)

SEQ ID NO: 1 - DNA, 20 bp, Artificial Sequence
  Forward Primer of Sox 9
agtacccgcatctgcacaac

SEQ ID NO: 2 - DNA, 20 bp, Artificial Sequence
  Reverse Primer of Sox 9
acgaagggtctcttctcgct

SEQ ID NO: 3 - DNA, 20 bp, Artificial Sequence
  Forward Primer of COL2A1
agcaaggtgaccagggtatt

SEQ ID NO: 4 - DNA, 18 bp, Artificial Sequence
  Reverse Primer of COL2A1
accaggagagccacgttc

SEQ ID NO: 5 - DNA, 20 bp, Artificial Sequence
  Forward Primer of ACAN
cgttgcagaccaggagcaat

SEQ ID NO: 6 - DNA, 20 bp, Artificial Sequence
  Reverse Primer of ACAN
ggtttggacgccacttctca

SEQ ID NO: 7 - DNA, 21 bp, Artificial Sequence
  Forward Primer of MMP3
tcctgatgttggtggcttcag

SEQ ID NO: 8 - DNA, 21 bp, Artificial Sequence
  Reverse Primer of MMP3
tgtcttggcaaatccggtgta

SEQ ID NO: 9 - DNA, 21 bp, Artificial Sequence
  Forward Primer of MMP13
actaccatcctgcgactcttg

SEQ ID NO: 10 - DNA, 21 bp, Artificial Sequence
  Reverse Primer of MMP13
gtttgccagtcacctctaagc

SEQ ID NO: 11 - DNA, 20 bp, Artificial Sequence
  Forward Primer of NOS2
accttgttcagctacgcctt

SEQ ID NO: 12 - DNA, 20 bp, Artificial Sequence
  Reverse Primer of NOS2
cattcccaaatgtgcttgtc

SEQ ID NO: 13 - DNA, 21 bp, Artificial Sequence
  Forward Primer of GLS
aacgtcagatggtgtcatgct

SEQ ID NO: 14 - DNA, 20 bp, Artificial Sequence
  Reverse Primer of GLS
tgaatttggccagctgagga

SEQ ID NO: 15 - DNA, 20 bp, Artificial Sequence
  Forward Primer of SLC1A5
tcgtctttggtgtggctctg

SEQ ID NO: 16 - DNA, 20 bp, Artificial Sequence
  Reverse Primer of SLC1A5
ctggcggacgtctttcatct

SEQ ID NO: 17 - DNA, 20 bp, Artificial Sequence
  Forward Primer of SLC7A5
cttcgccacctacttgctca

SEQ ID NO: 18 - DNA, 20 bp, Artificial Sequence
  Reverse Primer of SLC7A5
ccttgtcccatgtccttccc

SEQ ID NO: 19 - DNA, 21 bp, Artificial Sequence
  Forward Primer of SLC38A2
attgtgggcagtggaatcctt

SEQ ID NO: 20 - DNA, 20 bp, Artificial Sequence
  Reverse Primer of SLC38A2
tcgttggcagtcttgaggag

SEQ ID NO: 21 - DNA, 20 bp, Artificial Sequence
  Forward Primer of GS
ctgggttgatggtaccggag

SEQ ID NO: 22 - DNA, 20 bp, Artificial Sequence
  Reverse Primer of GS
cggaaggggtctcgaaacat

SEQ ID NO: 23 - DNA, 20 bp, Artificial Sequence
  Forward Primer of GLUD1
caatgcgcatgcctgtgtta

SEQ ID NO: 24 - DNA, 20 bp, Artificial Sequence
  Reverse Primer of GLUD1
catagagtgcaggcccacat

SEQ ID NO: 25 - DNA, 20 bp, Artificial Sequence
  Forward Primer of IDH1
cagagctctcttggaccgac

SEQ ID NO: 26 - DNA, 20 bp, Artificial Sequence
  Reverse Primer of IDH1
tgcagcatctttggtgacct

SEQ ID NO: 27 - DNA, 20 bp, Artificial Sequence
  Forward Primer of IDH2
aagtcttccggtggctttgt

SEQ ID NO: 28 - DNA, 20 bp, Artificial Sequence
  Reverse Primer of IDH2
tggtgttctcggtaatggcg

SEQ ID NO: 29 - DNA, 20 bp, Artificial Sequence
  Forward Primer of GOT1
agagcagtggaagcagatcg

SEQ ID NO: 30 - DNA, 20 bp, Artificial Sequence
  Reverse Primer of GOT1
aatagcgaatagcccacgca

SEQ ID NO: 31 - DNA, 20 bp, Artificial Sequence
  Forward Primer of GOT2
ggacctccagatcccatcct

SEQ ID NO: 32 - DNA, 23 bp, Artificial Sequence
  Reverse Primer of GOT2
ggttttccgttatcatcccggta

SEQ ID NO: 33 - DNA, 22 bp, Artificial Sequence
  Forward Primer of CCBL2
ttcaaaaacgccaaacgaatcg

SEQ ID NO: 34 - DNA, 21 bp, Artificial Sequence
  Reverse Primer of CCBL2
gatgaccaaagcctcttgtgt

SEQ ID NO: 35 - DNA, 23 bp, Artificial Sequence
  Forward Primer of GPT2
aaccattcactgaggtaatccga

SEQ ID NO: 36 - DNA, 23 bp, Artificial Sequence
  Reverse Primer of GPT2
gggctgtttagtaggtttgggta

SEQ ID NO: 37 - DNA, 19 bp, Artificial Sequence
  Forward Primer of IDH3a
tgggtgtccaaggtctctc

SEQ ID NO: 38 - DNA, 22 bp, Artificial Sequence
  Reverse Primer of IDH3a
ctcccactgaataggtgctttg

SEQ ID NO: 39 - DNA, 21 bp, Artificial Sequence
  Forward Primer of IDH3b
tggagaggtctcggaacatct

SEQ ID NO: 40 - DNA, 21 bp, Artificial Sequence
  Reverse Primer of IDH3b
agccttgaacacttccttgac

SEQ ID NO: 41 - DNA, 19 bp, Artificial Sequence
  Forward Primer of IDH3g
ggtgctgcaaaggcaatgc

SEQ ID NO: 42 - DNA, 21 bp, Artificial Sequence
  Reverse Primer of IDH3g
tatgccgcccaccatacttag